(19) United States
(12) Patent Application Publication
(10) Pub. No.: US 2010/0323040 A1
(43) Pub. Date: Dec. 23, 2010

(54) COSMETIC COMPOSITION FOR TOPICAL USE COMPRISING WASABI

(76) Inventor: Seri NOMURA, Paris (FR)

Correspondence Address: YOUNG & THOMPSON, 209 Madison Street, Suite 500, Alexandria, VA, 22314, US

(21) Appl. No.: 12/304,906

(22) PCT Filed: Jun. 18, 2007
(86) PCT No.: PCT/FR2007/001007

§ 371 Date: Sep. 18, 2009

(30) Foreign Application Priority Data
Jun. 16, 2006 (FR) 06 05402

(51) Int. Cl. A61K 8/97; A61Q 19/00

(52) U.S. Cl. 424/729; 424/755; 424/744; 424/750; 424/736

(57) ABSTRACT

The present invention relates to a cosmetic composition comprising an extract of wasabi, and a cosmetically acceptable carrier.

The present invention relates to a cosmetic composition based on wasabi, which can be used especially to moisturize, protect, repair, rejuvenate and beautify the skin.

Wasabi, also called “Wasabia japonica (Miq.) Matsum” or also known as Japanese horseradish, is a plant of the Brassicaceae family, a sub-family of the Brassicoideae, which is native to Japan.

Wasabi is a Japanese herb which grows in rivers, or in tanks especially designed for the purpose. In Japan, authentic wasabi is cultivated only in very pure water.

Wasabi is known for its antiseptic and detoxifying properties. It cures and prevents the occurrence of certain diseases; it strengthens the teeth and also prevents food poisoning. Wasabi also has anti-inflammatory qualities and acts as an anti-bacterial and anti-infectious agent.

The inventors have now demonstrated that wasabi also has especially advantageous cosmetic properties.

The subject-matter of the present invention is therefore a cosmetic composition comprising wasabi and a cosmetically acceptable carrier.

The expression “cosmetically acceptable carrier” means a carrier suitable for use in contact with human and animal cells, in particular the cells of the epidermis, without toxicity, irritation, undue allergic response and the like, and commensurate with a reasonable advantage/risk relationship.

Water, especially distilled water, may be mentioned as an example of a cosmetologically acceptable carrier.

In the context of the present description, the term “wasabi” encompasses, in particular, wasabi extracts. “Wasabi extract” means any type of substance and/or molecule obtained by aqueous and/or alcoholic extraction.

Preferably, a wasabi extract obtained by alcoholic extraction, for example with sake, is used.

Preferably, the cosmetic compositions described are natural. This term is intended to denote cosmetic compositions comprising 95%, preferably 98%, and especially 99% by weight, expressed relative to the total weight of the composition, of ingredients of natural origin.

Examples of products of natural origin are especially plant or tree extracts, especially vegetable oils, and water.

The expression “ingredients of natural origin” means ingredients which are found as such in nature or which are converted in accordance with techniques that are referred to as natural, i.e. that do not use non-natural chemical reagents, such as fermentation, extraction or distillation. These natural compounds may originate especially from organic farming.

Preferably, at least 5% by weight of the total of the ingredients originate from organic farming. Preferably, at least 50% of the vegetables used originate from organic farming. Preferably, the cosmetic compositions according to the invention comply with Ecocert® certification standards.

Preferably, the compositions contain less than 5% by weight, relative to the total weight of the composition, of synthetic chemical products, such as chemical preservatives or additives.

The expression “synthetic chemical products” means non-natural products, i.e. which do not occur naturally and/or which are synthesized chemically in accordance with a process comprising the use of non-natural chemical reagents or solvents.

In particular, synthetic products such as parabens, silicones, glycols, phenoxyethanol, colouring agents or perfumes produced synthetically, animal raw materials, mineral oils stemming from petrochemistry or raw materials comprising genetically modified organisms (GMO) are preferably absent from the compositions.

The cosmetic compositions may also comprise other ingredients, preferably originating from plants.

According to one embodiment, the cosmetic composition contains a wasabi extract and sake in combination.

It is preferable to use a wasabi extract obtained by extraction with sake.

Sake is a traditional drink which has been consumed in Japan for a long time. It first appeared in Japanese myth, and the modern method of preparing it has been in existence since the seventeenth century. It is a translucent and almost colourless drink which is obtained by fermenting rice and which contains an average of 15% alcohol.

Sake brings lasting moisturization to the skin. Owing to its high degree of purity, sake is a good alternative to numerous modern lifting products which contain harmful chemical components.

Preferably, the ratio of wasabi/sake by weight ranges from 1% to 5%, preferably from 2% to 4%, and more preferably from 2.5% to 3.5%.

Preferably, a sake whose alcohol content is from 10% to 20% by weight is used.

Preferably, sake represents from 10% to 80% by weight of the composition, more preferably from 15% to 75% by weight, in particular from 20% to 30% by weight, relative to the total weight of the composition.

According to another embodiment of the invention, the cosmetic compositions also comprise aloe vera.

Aloe vera (CTFA name: Aloe barbadensis) may be composed of active material to the extent of 100% and may be in the form of a powder or in the form of an extract. The expression “aloe vera extract” means any type of substance and/or molecule obtained by aqueous and/or alcoholic extraction.

Aloe vera is particularly useful as a natural moisturizing agent and as an anti-inflammatory agent for soothing the skin.

Preferably, aloe vera represents from 10% to 30% by weight, especially from 15% to 25% by weight, relative to the total weight of the composition.

The compositions according to the invention may also comprise the compounds normally used for the preparation of cosmetic compositions.

In particular, they may contain oils of vegetable, animal or mineral origin, natural or synthetic waxes and the various other fatty substances, provided of course that they are cosmetically acceptable.

Of the oils that may form part of the composition of the cosmetic compositions according to the invention, special mention may be made of:
mineral oils, such as paraffin oil and vaseline oil;
oils of animal origin, such as perhydrosqualene;
oils of vegetable origin, such as sweet-almond oil, avocado oil, castor oil, coriander oil, olive oil, jojoba oil, sesame oil, peanut oil, grapeseed oil, colza oil, copra oil, hazelnut oil, macadamia nut oil, shea butter, palm oil, apricot kernel oil, calophyllum oil, rice bran oil, corn oil, wheatgerm oil, soya bean oil, sunflower oil, onager oil, safflower oil, passion flower oil, rye oil, camellia oil (Camellia kissi), citrus lemon peel oil [Citrus limon(lime)], patchouli leaf oil (Pogostemon patchouli), and caprylic/capric acid triglycerides;
synthetic oils, such as purcellin oil, fatty acid esters, such as butyl myristate, isopropyl myristate, cetyl myristate, isopropyl palmitate, 2-ethylhexyl palmitate, isopropyl adipate, ethylhexyl adipate, butyl stearate, hexadecyl stearate, isopropyl stearate, octyl stearate, isocetyl stearate, decyl oleate, hexyl laurate, propylene glycol dicaprylate and the esters derived from lanolic acid, such as isopropyl lanolate, isocetyl lanolate, dioctylcyclohexane, isoparaffins and poly-α-olefins.

The term “oil” means a substance which is liquid at ambient temperature, unctuous, combustible and water-immiscible. In the above list, the oils predominantly comprise carbon atoms and hydrogen atoms, and optionally ester, ether, fluorinated, carboxylic acid and/or alcohol groups.

One oil or a mixture of several of the oils indicated above may be used.

According to a preferred variant of the invention, the oils forming part of the composition of the cosmetic formulations according to the invention are selected from oils of vegetable origin.

Preferably, the vegetable oils are selected from sheer butter, jojoba oil or jojoba butter, macadamia nut oil, rice bran oil, camellia seed oil (Camellia kissi), and sweet-almond oil.

The cosmetic compositions according to the invention may also comprise one or more natural extract(s) or essential oils(s) of plants, especially flowers, fruits or aromatic trees.

Plant extracts which may be mentioned are extracts of rosemary leaves; Lonerica japonica flowers; calendula flowers; liquorice; green tea (Camellia sinensis); rose; Turkish rose; Bulgarian rose; lavender; citrus citron, or yuzu; chamomile; patchouli leaf; mint; hamamelis; cucumber; vetiver; Hinoki wood; sandalwood; and cedar. The following ingredients are particularly preferred: Hinoki, sandalwood and cedar extracts; rose extracts; citrus extracts, especially Citrus junos and Citrus limon; aloe vera extracts.

The plant extracts have advantageous cosmetic qualities.

Green tea extract is a powerful anti-oxidant used to combat free radicals and protect and restore the skin's natural defense system. It is particularly useful in reducing the appearance of dark patches and whitening the skin.

Extract of Hinoki, more especially Japanese Hinoki, is an anti-bacterial agent and a natural astringent. It is particularly useful in closing the pores and regulating greasy skin.

Natural tree extracts, in particular Japanese Hinoki, sandalwood and cedar extracts, are therefore particularly useful in balancing and harmonizing combination skin.

Citrus extracts, in particular Japanese yuzu citron extracts, are particularly useful in refreshing the skin and giving it a matte appearance. They are generally suitable for all skin types.

The proportion of natural extracts or essential oils in the composition may vary to a great extent, depending on the purpose for which it is intended. Preferably, it represents from 1% to 10% by weight, especially from 2% to 6% by weight relative to the total weight of the composition.

The compositions according to the invention may also comprise proteins of vegetable origin such as soya proteins or polysaccharides such as hyaluronic acid or the cosmetically acceptable salts thereof.

The compositions according to the invention may also comprise one or more of the additives normally used in cosmetic formulation, such as sequestering agents, anti-oxidants, preservatives, fillers, electrolytes, humectants, colouring agents, mineral or organic acids or bases, moisturizers, vitamins, essential fatty acids, sphingolipids, self-tanning compounds, agents for soothing and protecting the skin, emulsifiers, hydrophilic or lipophilic gelling agents and sun filters.

These additives are used in the proportion conventionally employed, especially from 0.001% to 20% by weight relative to the total weight of the composition.

Preferably, the additives forming part of the composition of the formulations of the invention are selected from products of natural origin. An example of a natural preservative which may be mentioned is potassium sorbate or Lonicera japonica extracts.

Preferably, the cosmetic composition according to the invention is free from additives of non-natural origin. When it is necessary to add such an additive, the proportion of the additive of non-natural origin will advantageously be limited to that which is strictly necessary.

Depending on their nature, the adjuvants may be introduced into the fatty phase, into the aqueous phase and/or into the lipid vesicles.

A cosmetic composition according to the invention is preferably applied topically to the skin, including the mucosa, especially the lips, and the superficial body growths, especially the nails and the hair.

The cosmetic compositions according to the invention may be presented in a large number of galenical forms, especially a salve, cream, oil, milk, ointment, powder, impregnated pad, solution, gel, spray, lotion, suspension, soap or shampoo.

In particular, the cosmetic compositions may be presented in the form of a soap, for beauty and hygiene care for the body, a cream for face care (day cream or night cream), a face mask, an exfoliant for the face or body, a milk or lotion for cleansing or for removing make-up, or a moisturizing milk for the face or body.

The composition may be in a single-phase form, for example a solution, or may comprise several phases. In the latter case, it may be a simple oil-in-water or water-in-oil emulsion, a multiple emulsion, a microemulsion, a micellar solution, a gel, or a foam.

The cosmetic compositions of the invention are especially useful in moisturizing, soothing, repairing and/or protecting the skin from external attack, such as, especially, ultraviolet radiation or air pollution. They are advantageous in combating skin ageing, especially the phenomena of wrinkles, and loss of tonicity and elasticity due to structural changes in the skin caused by age.

The following Examples illustrate the invention without limiting the scope thereof.

### EXAMPLES

### Example 1

### Lotion for the Face

The phases A, B, C and D are prepared separately by mixing the compounds indicated in Table 1 in a suitable receptacle. Phase A is then added to phase B with agitation. Phase C is then introduced into phases A and B with moderate agitation until a homogeneous dispersion is obtained. Phase D is then introduced with agitation. Finally, phase E is added with agitation. A milky liquid having a slightly woody perfume and a pH of 6 is obtained.

TABLE 1

| | | % by |
|---|---|---|
| Phase | Ingredient | weight |
| A | Wood essences (Japanese Hinoki, sandalwood, | 0.93 |
| | cedar), | |
| | Composition of essential oils | 0.44 |
| | Water (and) filtrate of fermented rice | 25 |
| | (Japanese sake) and Japanese wasabi (Wasabia | |
| | japonica matsum) | |
| B | 10-polyglyceryl laurate | 3 |
| C | Water | 49.3 |
| | Extract of Aloe barbadensis leaves (and) | 20 |
| | water | |
| D | Maltodextrin, and liquorice and green tea | 1 |
| | extracts | |
| E | Potassium sorbate | 0.4 |

### Example 2

### Lotion for the Face

A composition is prepared from the mentioned compounds in the respective proportions indicated in Table 2, in the same manner as in Example 1. An orange-pink liquid having a slightly flowery perfume and a pH of 6 is obtained.

TABLE 2

| | | % by |
|---|---|---|
| Phase | Ingredient | weight |
| A | Composition of rose extracts | 1.08 |
| | Composition of essential oils | 0.23 |
| | Water (and) filtrate of fermented rice | 25 |
| | (Japanese sake) and Japanese wasabi (Wasabia | |
| | japonica matsum) | |
| B | 10-polyglyceryl laurate | 3 |
| C | Water | 35.29 |
| | Distilled water of Rosa damascena | 10 |
| | Water (and) extract of Aloe barbadensis | 20 |
| | leaves | |
| | Glycerin | 4 |
| D | Maltodextrin and extracts of Rosa Gallica | 1 |
| | flowers | |
| E | Potassium sorbate | 0.4 |

### Example 3

### Lotion for the Face

A composition is prepared from the ingredients indicated in Table 3 in the same manner as in Example 1. A yellow-green liquid having a slightly lemony perfume and a pH of 6.25 is obtained.

TABLE 3

| | | % by |
|---|---|---|
| Phase | Ingredients | weight |
| A | Extract of Citrus junos (Yuzu) and lime | 1.15 |
| | Water (and) filtrate of fermented rice | 25 |
| | (Japanese sake) and Japanese wasabi (Wasabia | |
| | japonica matsum) | |
| B | 10-polyglyceryl laurate | 3 |
| C | Water | 49.55 |
| | Water (and) extract of Aloe barbadensis | 20 |
| | leaves | |
| D | Maltodextrin and plant extracts | 1 |
| E | Potassium sorbate | 0.3 |

### Example 4

### Lotion for the face

### a) Preparation of Phase I

Phases A, B and C are prepared from the ingredients indicated in Table 4A below. The ingredients of phase B are then mixed with phase A in a suitable recipient with moderate agitation. The mixture obtained is then added to phase C with moderate agitation until a homogeneous dispersion is obtained.

b) Preparation of phase II

Phases A, B and C are prepared from the ingredients indicated in Table 4B below. B is then heated to 60° C. and A is subsequently introduced into B with moderate agitation. D is then introduced at less than 40° C. with moderate agitation. Finally, E is introduced.

### c) Preparation of the Gel

The two phases I and II obtained are combined with agitation. A pale green gel having a slightly woody perfume is obtained. The pH is 6.

TABLE 4A
Phase 1

| Phase | Ingredients | % by |
|---|---|---|
| | | weight |
| A | Essence of Chamaecyparis obtuse (Japanese | 0.1 |
| | Hinoki) | |
| B | Water (and) filtrate of fermented rice | 15 |
| | (Japanese sake) and Japanese wasabi (Wasabia | |
| | japonica matsum) | |
| C | 10-polyglyceryl laurate | 1 |
| | Water | 1 |

TABLE 4B
Phase II

| | | % by |
|---|---|---|
| Phase | Ingredients | weight |
| A | Sceloritum gum | 1.2 |
| B | Water | qs 100 |
| | Water (and) extract of Aloe barbadensis | 30 |
| | leaves and distilled water of hamamelis | |
| | Glycerin | 4 |
| | Potassium sorbate | 0.3 |
| D | Extracts of green tea, liquorice and lavender | 3 |
| E | Sodium hyaluronate | 0.06 |

1-9. (canceled)

10. Cosmetic composition comprising a wasabi extract and a cosmetically acceptable carrier.

11. Cosmetic composition according to claim 10, also comprising sake.

12. Composition according to claim 10, wherein the ratio of wasabi extract/sake by weight ranges from 1% to 5% by weight.

13. Composition according to claim 10, wherein the wasabi extract is obtained by extraction with sake.

14. Composition according to claim 10, also comprising aloe vera.

15. Cosmetic composition according to claim 10, also comprising one or more plant extracts selected from rosemary leaves; Lonerica japonica flowers; calendula flowers; liquorice; green tea; rose; Turkish rose; Bulgarian rose; lavender; citrus citron, or yuzu; chamomile; patchouli leaf; mint; hamamelis; cucumber; vetiver; Hinoki wood; sandalwood; and cedar.

16. Cosmetic composition according to claim 10, also comprising one or more vegetable oils selected from Shea butter, jojoba oil or jojoba butter, macadamia oil, rice bran oil, camellia seed oil and sweet-almond oil.

17. Composition according to claim 10, wherein the products of natural origin represent at least 95% by weight relative to the total weight of the composition.

18. Cosmetic composition according to claim 10, presented in the form of a salve, cream, oil, milk, ointment, powder, impregnated pad, solution, gel, spray, lotion, suspension, soap or shampoo.
